(19) United States
(12) Patent Application Publication
(10) Pub. No.: US 2023/0111264 A1
(43) Pub. Date: Apr. 13, 2023

(54) IDENTIFICATION OF AIR QUALITY OF INDOOR SPACES

(71) Applicant: Carrier Corporation, Palm Beach Gardens, FL (US)

(72) Inventors: Ganesh Kumar Malthurkar, Hyderabad (IN); Ramesh Lingala, Hyderabad (IN)

(21) Appl. No.: 17/962,856

(22) Filed: Oct. 10, 2022

Related U.S. Application Data
(60) Provisional application No. 63/254,628, filed on Oct. 12, 2021.

(51) Int. Cl. G01N 33/00; G01N 15/06; F24F 11/64; F24F 11/52; G05B 19/042

(52) U.S. Cl. CPC G01N 33/0075; G01N 15/06; F24F 11/64; F24F 11/52; G05B 19/042; F24F 2110/50

(57) ABSTRACT

A system and method for monitoring and identifying air quality for indoor spaces. A method includes receiving sensor data from a plurality of sensors, each of the plurality of sensors are associated with corresponding locations of an indoor space, and determining air quality for each of the locations of the indoor space. The method includes identifying each of the locations exceeding an air quality threshold based at least in part on comparing the sensor data to the air quality threshold, and transmitting a notification to one or more devices, wherein the notification includes air quality information for each of the locations.

### CROSS-REFERENCE TO RELATED APPLICATIONS

This application claims the benefit of Provisional Application No. 63/254,628 filed Oct. 12, 2021, the disclosure of which is incorporated herein by reference in its entirety.

### BACKGROUND

The present disclosure relates to control systems, and more specifically, to the identification and management of the air quality of indoor spaces.

People spend the majority of their time indoors and are therefore subject to the air quality in those spaces. This can include time spent in public buildings such as office spaces and retail establishments, and private spaces such as their homes. Other spaces can include manufacturing plants that can generate different levels of particulates that can the air quality. Often times, no thought is given to the air quality within the spaces that are visited most. The air quality can be impacted by a number of factors and can further impact one's health. For example, various materials and products such as building materials, cleaners, solvents, etc. may escape from the product and enter the air creating an unhealthy environment. The air quality can affect many health issues such as asthma, lung issues, irritate eyes, nose, throat, and more. Thus, there may be a need for a real-time air quality monitoring system to avoid such consequences.

### BRIEF DESCRIPTION

According to an embodiment, a method for intelligently monitoring and identifying air quality for indoor spaces is provided. The method can include receiving sensor data from a plurality of sensors, wherein each of the plurality of sensors are associated with corresponding locations of an indoor space, and determining air quality for each of the locations of the indoor space. The method can also include identifying each of the locations exceeding an air quality threshold based at least in part on comparing the sensor data to the air quality threshold, and transmitting a notification to one or more devices, wherein the notification includes air quality information for each of the locations.

In addition to one or more of the features described herein, or as an alternative, further embodiments include receiving access control system information to determine a number of people in a location corresponding to a card swipe or inputting credentials to an access lock or access reader.

In addition to one or more of the features described herein, or as an alternative, further embodiments include controlling access to each of the locations based on the determined air quality for each of the locations exceeding the air quality threshold.

In addition to one or more of the features described herein, or as an alternative, further embodiments include controlling a ventilation system to control air flow in the one or more each of the locations based on exceeding the air quality threshold.

In addition to one or more of the features described herein, or as an alternative, further embodiments include identifying available locations for a reservation system based on the determined air quality for each of the locations.

In addition to one or more of the features described herein, or as an alternative, further embodiments include using the air quality for each of the locations that is based at least in part on a number of people present users in each of the locations.

In addition to one or more of the features described herein, or as an alternative, further embodiments include predicting conditions of the air quality for each of the locations based at least in part on a number of expected people for each of the locations.

According to an embodiment, an intelligent monitoring system to monitoring and identify air quality for indoor spaces is provided. The system can include a plurality of sensors, and an intelligent monitoring system coupled to the plurality of sensors. The intelligent monitoring system can be configured to receive sensor data from the plurality of sensors, wherein each of the plurality of sensors are associated with corresponding locations of an indoor space, and determine air quality for each of the locations of the indoor space. The intelligent monitoring system can also be configured to identify each of the locations exceeding an air quality threshold based at least in part on comparing the sensor data to the air quality threshold, and transmit a notification to one or more devices, wherein the notification includes air quality information for each of the locations.

In addition to one or more of the features described herein, or as an alternative, further embodiments include an access control system that is configured to determine a number of people in a location corresponding to receiving a card swipe or inputting credentials to access lock or access reader.

In addition to one or more of the features described herein, or as an alternative, further embodiments include controlling the access control system to control each of the locations based on the determined air quality for each of the locations exceeding the air quality threshold.

In addition to one or more of the features described herein, or as an alternative, further embodiments include a ventilation system that is operable to control air flow in each of the locations based on exceeding the air quality threshold.

In addition to one or more of the features described herein, or as an alternative, further embodiments include an intelligent monitoring system that is configured to identify available locations for a reservation system based on the determined air quality for each of the locations.

In addition to one or more of the features described herein, or as an alternative, further embodiments include determining the air quality for each of the locations based at least in part on a number of people present in each of the locations.

In addition to one or more of the features described herein, or as an alternative, further embodiments include an intelligent monitoring system that is configured to predict conditions of the air quality for each of the locations based at least in part on a number of expected people for each of the locations.

In addition to one or more of the features described herein, or as an alternative, further embodiments include transmitting updates on the air quality to the one or more devices in real-time.

The foregoing features and elements may be combined in various combinations without exclusivity, unless expressly indicated otherwise. These features and elements as well as the operation thereof will become more apparent in light of the following description and the accompanying drawings. It should be understood, however, that the following description and drawings are intended to be illustrative and explanatory in nature and non-limiting.

### BRIEF DESCRIPTION OF THE DRAWINGS

The following descriptions should not be considered limiting in any way. With reference to the accompanying drawings, like elements are numbered alike:

FIG. 1 illustrates an example system for practicing the techniques of one or more embodiments of the disclosure described herein;

FIG. 2 illustrates an example monitoring system used in FIG. 1 that uses analytics to process the data in accordance with one or more embodiments of the disclosure;

FIG. 3 illustrates an example air quality meter for indoor spaces in accordance with one or more embodiments of the disclosure; and

FIG. 4 illustrates a flowchart of a method for identifying air quality of indoor spaces in accordance with one or more embodiments of the disclosure.

### DETAILED DESCRIPTION

The problem of identifying and managing air quality of closed premises is solved by providing an intelligent monitoring system that uses sensor data to make a determination on the air quality. By implementing the intelligent monitoring system, it is configured to reduce the processing of external systems by centralizing the processing. In addition, instead of various systems, separately and independently, processing all of the different source of data, the intelligent monitoring system can reduce the amount of processing and provide optimal control over the monitored spaces.

In one or more embodiments of the disclosure, an intelligent monitoring system processes multiple data points from various sensors to monitor and locate indoor spaces that have good air quality. Example sensors can include but are not limited to smoke detectors, humidity sensors, temperature sensors, and ambient air pressure sensors. In addition, access readers, controllable locks, and thermostats can be used to capture various data for counting the number of users present in a location, performing space analysis of the location, controlling access to the location, etc. The detected air quality can be classified using global standards for air quality.

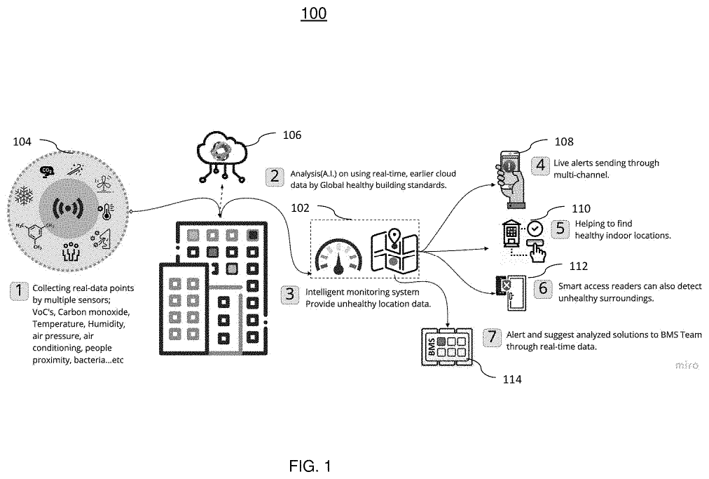

FIG. 1 depicts an example system 100 in accordance with one or more embodiments of the disclosure. The system 100 includes an intelligent monitoring system 102 that is configured to analyze the received data. The intelligent monitoring system 102 can be implemented in a server or other processing or computing device.

The system 100 also includes a plurality of sensors 104 that are coupled to the intelligent monitoring system 102. The intelligent monitoring system 102 can associate the location of the sensor with the detected sensor reading. The sensors 104 can include sensors that are capable of detecting various conditions such as volatile organic compounds (VOCs), carbon monoxide (C), carbon dioxide (CO2), dust particles/particulate matter (PM), temperature, humidity, air pressure, air conditioning, people proximity, bacteria, radiation, etc. In other embodiments, sensors can be used to detect various odors such as those that are harmful to people and the environment.

The intelligent monitoring system 102 can also be coupled to a cloud 106 that is configured to analyze the data. The cloud 106 can implement an artificial intelligence (AI) service to analyze the real-time data from the sensors, other sources, and process the real-time data with historical data for the detected sensor measurements. In some embodiments, the cloud or cloud service can compare the real-time data to data indicative of the global health standards for air quality.

In one or more embodiments of the disclosure, the intelligent monitoring system 102 can be integrated with a plurality of different systems/devices. The intelligent monitoring system 102 can also be coupled to a plurality of user devices 108 that can be configured to receive notifications from the intelligent monitoring system 102 indicating at least one suitable indoor location 110 or the healthiest rated indoor space. In other embodiments, the intelligent monitoring system 102 can be integrated with a user credentialing device that can be used to obtain and process data for each space and trigger an event to a building management system (BMS) or administrator regarding the health status of the locations. The intelligent monitoring system 102 can be configured to exchange messages between user devices that are internally connected to a building network and those that are outside of the network by using a mobile application interface. The mobile application interface may be used to allow user devices to directly share information regarding the health status of the spaces with other user devices.

As shown in FIG. 1, an external system can include a smart access system 112. In one or more embodiments of the disclosure, smart access readers can be associated with various locations of an indoor space. The smart access readers can also detect unhealthy surroundings and report the status to the intelligent monitoring system 102. The intelligent monitoring system 102 can associate the location of the access card reading to a count of a number of people associated with a defined space. In one or more embodiments of the disclosure, responsive to a user presenting credentials to the access readers (associated with one access to a particular location, zone, room, building, etc.), the intelligent monitoring system 102 can be configured to provide a message indicating the health status of a previously accessed area to the user device. The alert provided from the intelligent monitoring system 102 to a user device can include but is not limited to a voice command, a text alert (e.g., text message), a light indicator such as at an access reader, etc.

In addition, the intelligent monitoring system 102 can be configured to alert a building management team or provide a control signal to control smart devices (ventilation) or turn off one or more polluting devices, or deny access to locations that exceed a health status. In one or more embodiments, an LED display can be provided at various location to provide an indication of the current health status of the respective locations.

In one or more embodiments of the disclosure, the intelligent monitoring system 102 can identify conditions that encourage the growth of bacteria or other unwanted materials that can be detrimental to one's health. For example, the temperature and moisture of an area may indicate conditions that are ripe for bacteria growth. In one or more embodiments of the disclosure, the intelligent monitoring system 102 can be configured to generate reports indicating a health or hazard report.

One or more illustrative embodiments of the disclosure are described herein. Such embodiments are merely illustrative of the scope of this disclosure and are not intended to be limiting in any way. Accordingly, variations, modifications, and equivalents of embodiments disclosed herein are also within the scope of this disclosure.

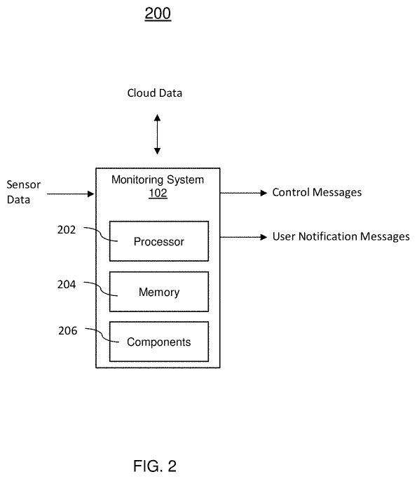

FIG. 2 depicts components of an example intelligent monitoring system 102 in accordance with one or more embodiments of the disclosure. The intelligent monitoring system 102 includes a processor 202 and a memory 204 to carry out the operations for performing intelligent monitoring. It can be appreciated the intelligent monitoring system 102 can include other components or modules and is not limited by the components shown in FIG. 2. In one or more embodiments of the disclosure, the processor 202 can include a processor of a general-purpose computer, special purpose computer, or other programmable data processing apparatus configured to execute instruction via the processor of the computer or other programmable data processing apparatus. The memory 204 can include any one or combination of volatile memory elements (e.g., random access memory (RAM, such as DRAM, SRAM, SDRAM, etc.)) and nonvolatile memory elements (e.g., ROM, erasable programmable read only memory (EPROM), electronically erasable programmable read only memory (EEPROM), etc.). It can be appreciated the intelligent monitoring system can include other components 206 and is not limited by the illustration of FIG. 2.

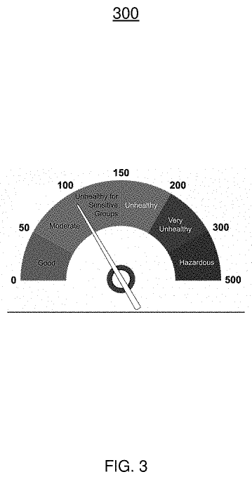

FIG. 3 depicts an example air quality meter 300 of results from an intelligent monitoring system 102 that can be provided to a user device or system to indicate the air quality that has been determined with one or more locations. It should be understood that this is an example illustration, and the classification of air quality can be defined by different ranges. As shown in FIG. 3, an intelligent monitoring system 102 that returns air quality meter 300 returning a value that is in the range of 0-50 can be classified as “Good.” The “Good” classification can represent air quality that has a minimal level of particulates or debris that has been detected by the sensors. Also, the number of particulates or debris can be impacted by the type and size of the particulates, the area of the defined space, the number of people in the defined space, the ventilation, the amount of particulate or debris producing sources, etc. For example, the air quality thresholds can be based on the number of current people that are present in the area. Also, the air quality threshold may be based on a prediction for the number of expected people for a defined space such as a conference that may occur in the future. Therefore, the intelligent monitoring system can adjust and/or prediction air quality conditions based on the number of people that are present or expected to be present. Other classifications can include a “Moderate,” “Unhealthy for Sensitive Groups,” “Unhealthy,” “Very Unhealthy,” and “Hazardous.” In a non-limiting example, the “Moderate” classification is defined in the range of 50-100, the “Unhealthy for Sensitive Groups” classification is defined in the range of 100-150, the “Unhealthy” classification is defined in the range of 150-200, the “Very Unhealthy” classification is defined in the range of 200-300, and the “Hazardous” classification is defined in the range of 300-500. Each of the classifications progressively include more and more particulates or debris as indicated by the value determined by the intelligent monitoring system 102. In one or more embodiments of the disclosure, the message is transmitted by the intelligent monitoring system 102 to the devices to display the air quality meter 300. In different embodiments of the disclosure, the message can a user map indicating a visual map-like representation of healthy areas or other non-healthy areas that have been determined by the intelligent monitoring system 102.

In some embodiments, the determined value or classification can be associated with various levels of alerts and controls. In a non-limiting example, the detected air quality for a location is between 150-200, the intelligent monitoring system 102 can begin transmitting the alerts to the connected devices and systems. The alarm level or threshold is configurable and can selected according to the type of location. In another example, the intelligent monitoring system 102 can provide controls to increase the ventilation of a ventilation system in a defined space if a configurable threshold level is reached. In another example, the intelligent monitoring system 102 can block or grant access to an area that is controlled by an access-controlled system based on a configurable threshold for air quality. In other examples, real-time alerts and notifications can be provided to user devices on a periodic basis or responsive to reaching one or more configurable thresholds. In one or more embodiments, the alerts or notifications may be provided using an audio or visual indicator in one or more locations that have detected the harmful condition. For example, the visual indicators can include the use of smart lighting to indicate the various warnings levels based on its color such as red or yellow. It can be appreciated that other controls and/or control systems can be managed by the intelligent monitoring system 102. The real-time alerts and notification can be transmitted to other devices, platforms or services and is not intended to be limited by the examples described herein. In some embodiments, users may be alerted to the health status of a location such as an office/building premises based on the user's previous access history to the location and proximity to the location. The user device may be used to display healthy work spaces and unhealthy work spaces in the office/building premises prior to reaching the location.

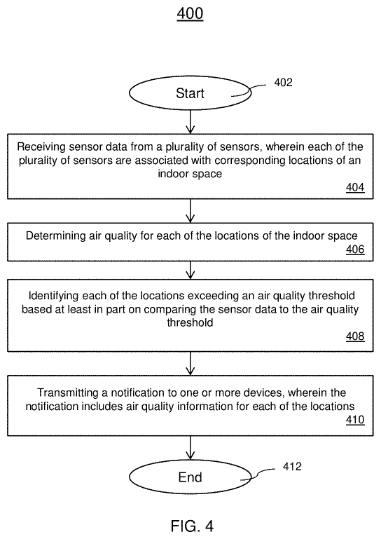

FIG. 4 depicts a flowchart of a method 400 for monitoring air quality of indoor spaces in accordance with one or more embodiments of the disclosure. The method 400 can be implemented in the system 100 shown in FIG. 1. The method 400 begins at block 402 and proceeds to block 404 which provides for receiving sensor data from a plurality of sensors, wherein each of the plurality of sensors are associated with corresponding locations of an indoor space. In one or more embodiments of the disclosure, the monitoring system 100 receives the sensor data. The sensors can be associated with corresponding locations by being arranged in a position to monitor a particular location. Each location can include one sensor or multiple sensors that are used to obtain data to monitor the air quality of each of the respective locations. Block 406 determines air quality for each of the locations of the indoor. In one or more embodiments of the disclosure, the air quality thresholds can be determined based on global standards for air quality. The air quality can be determined using the sensor data obtained from each location. For example, the number of detected particulates, the number of people within a defined space, the size of the defined space, etc. can all be used to determine a current air quality level for each space. Block 408 identifies each of the locations exceeding an air quality threshold based at least in part on comparing the sensor data to the air quality threshold. In one or more embodiments, if the value representing the current air quality for a location exceeds the value representing the air quality threshold for the corresponding locations, each of those locations can be identified as having bad air quality or low air quality. In one or more embodiments, each of the locations can have the same air quality threshold or each of the locations can be configured with different air quality thresholds. Block 410 transmits a notification to one or more devices, wherein the notification includes air quality information for each of the locations. The devices can include user devices such mobile phones, tablets, etc. It can be appreciated that notifications may be transmitting to other systems such as building management systems to increase the air flow of a ventilation system in low air quality areas or to access systems to lock/unlock areas based on a detected air quality. The method 400 ends at block 412. It should be understood that different steps or a different sequence of steps can be included and the method 400 is not intended to be limited by the steps shown.

The technical effects and benefits include identifying high air quality indoor spaces according to established global health standards. The technical effects and benefits can also include improving personal health, cognitive function and societal health by actively managing the air quality of the indoor spaces. In addition, the technical effects and benefits include improving the operation of one or more systems by centrally controlling the air quality and access to defined spaces based on the aggregated sensor data.

A detailed description of one or more embodiments of the disclosed apparatus and method are presented herein by way of exemplification and not limitation with reference to the Figures.

The term “about” is intended to include the degree of error associated with measurement of the particular quantity based upon the equipment available at the time of filing the application.

The terminology used herein is for the purpose of describing particular embodiments only and is not intended to be limiting of the present disclosure. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, element components, and/or groups thereof.

While the present disclosure has been described with reference to an exemplary embodiment or embodiments, it will be understood by those skilled in the art that various changes may be made and equivalents may be substituted for elements thereof without departing from the scope of the present disclosure. In addition, many modifications may be made to adapt a particular situation or material to the teachings of the present disclosure without departing from the essential scope thereof. Therefore, it is intended that the present disclosure not be limited to the particular embodiment disclosed as the best mode contemplated for carrying out this present disclosure, but that the present disclosure will include all embodiments falling within the scope of the claims.

What is claimed is:

1. A method for identifying air quality for indoor spaces, the method comprising:
receiving, at an intelligent monitoring system, sensor data from a plurality of sensors, wherein each of the plurality of sensors are associated with corresponding locations of an indoor space;
determining air quality for each of the locations of the indoor space;
identifying each of the locations exceeding an air quality threshold based at least in part on comparing the sensor data to the air quality threshold; and
transmitting a notification to one or more devices, wherein the notification includes air quality information for each of the locations.

2. The method of claim 1, further comprising receiving access control system information to determine a number of people in a location corresponding to a card swipe or inputting credentials to an access lock or access reader.

3. The method of claim 1, further comprising controlling access to each of the locations based on the determined air quality for each of the locations based on exceeding the air quality threshold.

4. The method of claim 1, further comprising controlling a ventilation system operable to control air flow in each of the locations based on exceeding the air quality threshold.

5. The method of claim 1, further comprising identifying available locations for a reservation system based on the determined air quality for each of the locations.

6. The method of claim 1, wherein the air quality for each of the locations is based at least in part on a number of people present in each of the locations.

7. The method of claim 1, further comprising predicting conditions of the air quality for each of the locations based at least in part on a number of expected people for each of the locations.

8. The method of claim 1, wherein transmitting the notification comprises transmitting updates on the air quality to the one or more devices in real-time.

9. A system for identifying air quality for indoor spaces, the system comprising:
a plurality of sensors;
an intelligent monitoring system coupled to the plurality of sensors, the monitoring system is configured to:
receive sensor data from the plurality of sensors, wherein each of the plurality of sensors are associated with corresponding locations of an indoor space;
determine air quality for each of the locations of the indoor space;
identify each of the locations exceeding an air quality threshold based at least in part on comparing the sensor data to the air quality threshold; and
transmit a notification to one or more devices, wherein the notification includes air quality information for each of the locations.

10. The system of claim 9, further comprising an access control system configured to determine a number of people in a location corresponding to receiving a card swipe or inputting credentials to access lock or access reader.

11. The system of claim 10, further comprising controlling the access control system to control to each of the locations based on the determined air quality for each of the locations based on exceeding the air quality threshold.

12. The system of claim 9, further comprising a ventilation system operable to control air flow in each of the locations based on exceeding the air quality threshold.

13. The system of claim 9, wherein the intelligent monitoring system is configured to identify available locations for a reservation system based on the determined air quality for each of the locations.

14. The system of claim 9, wherein the air quality for each of the locations is based at least in part on a number of people present in each of the locations.

15. The system of claim 9, wherein the intelligent monitoring system is configured to predict conditions of the air quality for each of the locations based at least in part on a number of expected people for each of the locations.

16. The system of claim 9, wherein the notification comprises transmitting updates on the air quality to the one or more devices in real-time.
